(19) United States
(12) Patent Application Publication
(10) Pub. No.: US 2024/0183001 A1
(43) Pub. Date: Jun. 6, 2024

(54) LIGNIN-BASED COMPOSITIONS AND RELATED HEAVY METAL RECOVERY METHODS

(71) Applicant: LignoSol IP Limited, San Gwann (MT)

(72) Inventors: Desmond Alexander SOMERVILLE, San Gwann (MT); Patrick Dieter WAIBEL, San Gwann (MT)

(21) Appl. No.: 18/285,697

(22) PCT Filed: Apr. 5, 2022
(86) PCT No.: PCT/IB2022/053162

§ 371 Date: Oct. 5, 2023

(30) Foreign Application Priority Data
Apr. 6, 2021 (GB) 2104869.9
Nov. 8, 2021 (GB) 2115987.6

(51) Int. Cl. C22B 3/18; C22B 3/24; C22B 11/00

(52) U.S. Cl. CPC C22B 3/18; C22B 3/24; C22B 11/00

(57) ABSTRACT

Compositions are provided for heavy or precious metal separation and/or recovery applications. In some embodiments, the composition comprises lignin, in particular technical lignin, and at least one strain of bacteria capable of biosurfactant production, and/or such biosurfactant. Also provided is a method for separating and/or recovering heavy or precious metals from a crushed or milled ore comprising the metals.

### CROSS-REFERENCE TO RELATED APPLICATIONS

This application claims priority to GB Provisional Patent Application No. 2104869.9, filed 6 Apr. 2021, and GB Provisional Patent Application No. 2115987.6, filed 8 Nov. 2021, the disclosures of which are hereby incorporated by reference in their entireties.

### BACKGROUND OF THE INVENTION

The present disclosure relates to the recovery of so-called heavy metals and precious metals from, for example, metal bearing ores. More particularly, the present disclosure relates to lignin-based compositions for heavy metal and precious metal recovery applications and related methods.

Mineral processing, such as beneficiation, for example, plays a key role in extractive metallurgy in which metals are extracted from their ores prior to being presented for refining and further treatment for end use requirements. In a typical ore body, valuable minerals including valuable metals are surrounded by gangue. The principle function of mineral processing is to liberate and concentrate the valuable minerals for further processing.

Typically the ore or rock is crushed or mechanically ground or milled in order to liberate or expose the valuable metals, also known as comminution. Various crushing and grinding/milling processes take place during comminution in order to generate course material suitable for screening and classification, the process by which coarser particles are separated or settled out from the finer particles. Once classified, the valuable metals are separated from the gangue and carrying medium, typically water, based on the specific gravity of the metals.

The separated metals are then concentrated and further processed for end use requirements.

In the case of certain high value or precious metals, such as gold, silver and the like, beneficiation is often applied directly on so-called run-of-mine ores, followed by extraction of the gold, silver or the like as a relatively pure metal. However, these processes often require the use of toxic chemicals that have a negative impact on the environment.

### SUMMARY OF THE INVENTION

In one aspect of the invention, there is provided a method for separating and/or recovering metals, in particular heavy metals or precious metals, from a crushed or milled ore material comprising the metals, the method comprising:
providing a composition comprising lignin, in particular technical lignin, and at least one isolated strain of bacteria capable of producing at least one biosurfactant, and/or at least one biosurfactant produced from at least one isolated strain of bacteria capable of producing a biosurfactant;
contacting the crushed or milled ore material with the composition such that at least a portion of the metals or metal containing material separate and settle out from the rest of the crushed or milled ore material; and
recovering the separated and settled metals or metal containing material.

In another aspect of the invention, there is provided a heavy metal separation and/or recovery composition suitable for separating and/or recovering heavy or precious metals from an ore, in particular crushed or milled ore, the composition comprising lignin, in particular technical lignin, and at least one isolated strain of bacteria capable of producing at least one biosurfactant, and/or at least one biosurfactant produced from at least one isolated strain of bacteria capable of producing a biosurfactant.

In some embodiments, the heavy metal separation and/or recovery composition further comprises a catholyte solution.

In some embodiments, the catholyte solution is a stabilized or upgraded catholyte solution.

Other aspects and features of the present invention will become apparent, to those ordinarily skilled in the art, upon review of the following description of specific embodiments of the disclosure.

### DESCRIPTION OF PREFERRED EMBODIMENTS

The heavy or precious metal separation and/or recovery compositions of the invention, in particular lignin-based compositions, are provided for heavy or precious metal separation and/or recovery applications, in particular for separation and/or recovering heavy or precious metals from a crushed or milled ore material comprising the metals.

As used herein, “heavy metals” refers to those metals that have a density or specific gravity sufficient for the metal bearing material to be separated from the gangue and the composition, typically by settling under gravity in one or more settling tanks. Examples of “heavy metals” include gold, platinum, palladium, silver, titanium, antimony, gallium, thallium and the like. By “precious metals” is understood to include those metals that may not strictly be regarded as heavy metals, but that nonetheless are capable of separation from a crushed or milled ore body in a similar manner. These include the platinum group metals osmium, iridium, rhodium, and the like.

As used herein, “lignin” refers to a biopolymer that is found in the secondary cell wall of plants and some algae. Lignin is a complex cross-linked phenolic polymer with high heterogeneity. Typical sources for the lignin include, but are not limited to, softwood, hardwood, and herbaceous plants such as corn stover, bagasse, grass, and straw, for example.

In some embodiments, the lignin comprises technical lignin. As used herein, “technical lignin” refers to lignin that has been isolated from lignocellulosic biomass, for example, as a byproduct of a pulp and paper production or a lignocellulosic biorefinery. Technical lignins may have a modified structure compared to native lignin and may contain impurities depending on the extraction process. In some embodiments, the technical lignin comprises at least one of Kraft lignin, lignosulfonates, soda lignin, organosolv lignin, steam-explosion lignin, and enzymatic hydrolysis lignin. In other embodiments, the technical lignin may comprise any other form of technical lignin.

In embodiments where the lignin comprises lignosulfonates, the lignosulfonates may be in the form of a salt including, for example, sodium lignosulfonate, calcium lignosulfonate, or ammonium lignosulfonate.

In other embodiments, the technical lignin is in the form of unhydrolyzed Kraft black liquor. Black liquor is a byproduct of the Kraft process and may contain not only lignin but hemicellulose, inorganic chemicals used in the pulping process, and other impurities. In other embodiments, the technical lignin is in the form of “brown liquor” (also referred to as red liquor, thick liquor and sulfite liquor) which refers to the spent liquor of the sulfite process. In other embodiments, the technical lignin may be in the form of any other spent cooking liquor of a pulping process or any other suitable lignin-based byproduct.

In other embodiments, the lignin may be synthetic lignin or any other suitable type of lignin.

In some embodiments, the lignin is hydrolyzed. As used herein, “hydrolyze” refers to using acid or base hydrolysis to at least partially separate lignin from the polysaccharide content of the lignocellulosic biomass. For example, where the lignin is in the form of black liquor, carbon dioxide may be used to precipitate Kraft lignin from the black liquor and then the Kraft lignin may be neutralized with sodium hydroxide.

In some embodiments, the lignin is in aqueous suspension. As used herein, an “aqueous suspension” of lignin refers to solid particles of lignin suspended, dispersed, and/or dissolved in a solvent that at least partially comprises water. In some embodiments, the solvent comprises substantially all water. In other embodiments, the solvent may comprise a combination of water and any other suitable solvent.

In some embodiments, the aqueous suspension of lignin may have a solids content of about 10% to about 90%, or about 25% to about 75%, or about 30% to about 60%, or about 33% to about 55%, or about 50% to about 60%. In some embodiments, the aqueous suspension of lignin may have a solids content of about 10% or above, or of about 25% or above, or of about 30% or above, or of about 33% or above, or of about 50% or above. In some embodiments, the aqueous suspension of lignin may have a solids content of about 90% or below, or of about 75% or below, or of about 60% or below, or of about 55% or below. In some embodiments, the aqueous suspension has a solids content of about 46%. A solids content of about 33% to about 55% may allow the composition to be flowable, which may be preferred for some applications. In other applications, the composition may be used as a slurry and the solids content may be as high as about 85% to about 90%.

In some embodiments, the lignin comprises at least one of lignin nanoparticles and lignin microparticles. As used herein, “nanoparticle” refers to a particle in the nanometer size range, for example, between about 1 nm and about 100 nm, and “microparticle” refers to a particle in the micrometer size range, for example, between about 100 nm and about 1000 μm (1 mm). In some preferred embodiments, the lignin particles have a size of about 200 nm or less, or about 100 nm or less. In some preferred embodiments, at least about 20%, or at least 30%, or at least 40%, or at least 50%, or at least 60%, or at least 70%, or at least 80%, or at least 90% of the lignin particles are nanoparticles having a size of about 100 nm or less.

The lignin nanoparticles and/or microparticles can be produced by any suitable method. For example, the lignin nanoparticles and/or microparticles can be produced using at least one of: solvent shifting; pH shifting; cross-linking polymerization; mechanical treatment; ice-segregation; template based synthesis; aerosol processing; electro spinning; and carbon dioxide (CO₂) antisolvent treatment. Such methods are described in Beisl et al. “Lignin from Micro- to Nanosize: Production Methods” Int. J. Mol. Sci. 2017; 18: 1244, incorporated herein by reference in its entirety.

In some preferred embodiments, lignin nanoparticles are produced using a pH shifting method, for example, as disclosed in Beisl et al. Briefly, the starting lignin material may be dissolved in a basic solution (e.g. an aqueous NaOH solution at pH 12) and the pH of the solution may be gradually decreased by addition of acid (e.g. HNO₃) to precipitate lignin nanoparticles. The solution may then be neutralized (e.g. by addition of NaOH) to resuspend the nanoparticles. The resulting particles may have a size of about 200 nm or less, or about 100 nm or less. In other embodiments, the lignin nanoparticles may be produced by any other suitable method.

By providing the lignin in the form of lignin nanoparticles and/or microparticles, the surface area of the lignin is increased, thereby also increasing the negative force around each particle. In addition, lignin nanoparticles and/or microparticles may have improved solubility in water. Conventional lignins are typically only soluble in water at alkaline pH; however, nanoparticles and/or microparticles may be soluble in approximately neutral water (Beisl et al.), which may be preferred for some applications.

In some embodiments, where the lignin comprises an aqueous suspension of lignin nanoparticles, the zeta potential value of the suspension may be about −5 to about −80 mV. In some embodiments, the specific gravity of the aqueous suspension of lignin nanoparticles is between about 1.286 to about 1.7 SG.

The composition further comprises at least one isolated strain of bacteria capable of producing at least one biosurfactant, and/or at least one biosurfactant produced from at least one bacteria capable of producing a biosurfactant.

As used herein, “isolated” or “isolate”, when used in reference to a strain of bacteria, refers to bacteria that have been separated from their natural environment. In some embodiments, the isolated strain or isolate is a biologically pure culture of a specific strain of bacteria. As used herein, “biologically pure” refers to a culture that is substantially free of other organisms.

As used herein, “biosurfactant” refers to compounds that are produced at the bacterial cell surface and/or secreted from the bacterial cell and function to reduce surface tension and/or interfacial tension. Non-limiting examples of biosurfactants include: lipopeptides, surfactin, glycolipids, rhamnolipids, methyl rhamnolipids, viscosin, and the like. The isolated strain may be capable of producing one or more types of biosurfactant.

In some embodiments, the isolated strain may produce one or more additional active compounds. For example, the isolated strain may produce a biopolymer, solvent, acid, exopolysaccharide, and the like.

In some embodiments, the at least one isolated strain of bacteria comprises a strain of Bacillus. In other embodiments, the at least one isolated strain comprises a strain of bacteria capable of biosurfactant production and that is non-pathogenic. Non-limiting examples of suitable strains are listed in Satpute et al. “Methods for investigating biosurfactants and bioemulsifiers: a review” Critical Reviews in Biotechnology, 2010, 1-18. For example, the at least one isolated strain of Bacillus may be Bacillus amyloliquefaciens, Bacillus licheniformis, Bacillus pumilus, Bacillus subtilis, or combinations thereof, in particular Bacillus licheniformis.

In some embodiments, the pH of the composition may be selected or adjusted to provide a suitable pH for the isolated strain(s). In some embodiments, the composition may further comprise one or more nutrients to support growth of the bacteria such as, for example, acetate, one or more vitamins, and the like.

In some embodiments, the isolated strain is in a viable form. For example, in some embodiments, the isolated strain may be in the form of a liquid suspension. In some embodiments, the isolated strain may be incubated for a suitable period of time prior to incorporation into the composition such that at least a portion of biosurfactant(s) are secreted into the bacterial suspension and therefore can be incorporated into the composition. For example, the bacteria can be incubated/fermented for between about one day and about six months or longer. The isolated strain may be incubated in the presence of a nutrient source and under suitable conditions (e.g. temperature, agitation, etc.) to produce the biosurfactant(s).

In other embodiments, the isolated strain may be in a lyophilized (freeze-dried) form. In some embodiments, the freeze-dried form comprises freeze-dried spores.

In some embodiments, where the isolated strain is in the form of a liquid suspension or in a freeze-dried form, the composition may comprise approximately 40 billion CFU (colony forming units) may be combined with at least about 1 g of lignin and up to several tons of lignin.

In other embodiments, the isolated strain may in an inviable form. For example, the isolated strain may be in the form of heat-killed cells or a cell lysate. In these embodiments, the bacteria of the isolated strain may be incubated for a suitable period of time prior to loss of viability (e.g. heat killing or lysis) such that a sufficient quantity of biosurfactant(s) is/are secreted into the bacterial suspension for incorporation into the composition. For example, the bacteria may be incubated for at least one week prior to loss of viability.

In other embodiments, a liquid suspension of bacteria may be incubated to produce the biosurfactant(s) and a supernatant containing the biosurfactant(s) may be separated from the bacterial cells and used in the composition.

Without being limited by theory, it is believed that the combination of lignin and the biosurfactant produced by the isolated strain act to mimic the natural habitat of the biosurfactant producing strains. The lignin may function as a growth substrate that contains required nutrients (carbon and fructose) to support growth of the bacteria, with the exception of additional acetate and metallic vitamins which may be added to the composition as needed.

In addition, a series of drop collapse tests were conducted to evaluate additional benefits of combining the lignin with a suitable biosurfactant in the composition of the invention. In particular, the tests were carried out to determine the effectiveness of the compositions of the invention in reducing the surface tension of water and other liquids. The results indicated that a further advantage in combining the lignin and biosurfactant in the composition of the invention is a significant reduction in surface tension at concentrations of between about 10 ppm and 300 ppm of the biosurfactant, which assists significantly in the compositions ability to cut through hydrocarbon containing materials.

In some embodiments, the lignin-based separation and/or recover compositions of the invention further comprise catholyte solutions. As used herein, “catholyte solution” is an activated solution produced in an electrochemical reaction, and is that part of the electrolyte solution adjacent the cathode of an electrochemical cell. It can be produced, for instance, from a 0.05%-1% salt brine (NaCl or KCl), and has a pH in the range 10.0 to 13.0 and an ORP/Redox value of less than about −800 mV, typically in the order of −900 to −950 mV. In the case of an NaCl starting solution, the active ingredient is highly active, and typically unstable, NaOH.

The separation and/or recovery compositions of the invention can comprise from about 1% to about 75% by volume of the catholyte solution.

In some embodiments, the composition further comprises at least one of a carboxylic acid or a salt or ester thereof. In some embodiments, the carboxylic acid is a di-carboxylic acid or a salt or ester thereof. The carboxylic acid or salt/ester thereof may function as a solvent, for example, by facilitating formation of a stable emulsion of the various components of the composition. In some embodiments, the composition comprises a carboxylic acid ester. In some embodiments, the carboxylic acid ester comprises a methyl ester or a butyl ester. In some embodiments, the butyl esters are produced by biochemical metathesis. In some embodiments, the butyl ester comprises n-Butyl 4-oxopentanoate. In some embodiments, the methyl ester comprises unsaturated C₁₀ or C₁₂ methyl ester. In some embodiments, the methyl ester comprises methyl 9-decenoate or methyl 9-dodecenoate. In some embodiments, the methyl ester is produced from a plant oil feedstock.

In other embodiments the di-carboxylic acid or a salt or ester thereof may comprise at least one oleic acid or a salt or ester thereof. In some embodiments, the oleic acid or a salt or ester thereof may be provided in the form of “tall oil”, a viscous liquid obtained as a byproduct of the Kraft process. In some embodiments, the tall oil may be distilled to tall oil rosin or tall oil fatty acid (TOFA) which comprise a higher proportion of oleic acids than tall oil.

Importantly, the carboxylic acid or salt or ester thereof acts as an anti-frothing agent, which is required to prevent any separated metals from being taken up in a froth that may otherwise form in the compositions of the invention, but rather to separate out and settle for recovery.

In other embodiments, the carboxylic acid may comprise acetic acid and/or pyroligneous acid, as described in more detail below.

In some embodiments, the composition comprises a combination of two or more carboxylic acids or salts/esters thereof. As one example, the composition may comprise a combination of one or more of: di-carboxylic acid, pyroligneous acid, and butyl esters produced by biochemical metathesis.

In some embodiments, the composition may comprise about 1% to about 30%, or about 1% to about 20%, or about 1% to 10% of di-carboxylic acid and/or pyroligneous acid and/or butyl esters by volume.

In some embodiments, the composition further comprises carbon black. The carbon black may be electroconductive carbon black and the carbon black may function to increase the conductivity of the composition. In some embodiments, the carbon black may be conductive, superconductive, extraconductive or ultraconductive carbon black. In some embodiments, the carbon black may be in the form of carbon black beads, microparticles, and/or nanoparticles. For example, the carbon black may comprise Printex™ XE2 B Beads from Orion Engineered Carbons™. In some embodiments, the composition may comprise about 0.5% to about 10% carbon black by volume. In some embodiments, addition of carbon black may increase the negative zeta potential of the composition thereby increasing its electrical stability. In other embodiments, the composition may comprise any other highly conductive microparticle and/or nanoparticle.

Optionally, the composition may further comprise pyrolysis oil. Pyrolysis oil may also be referred to as wood oil. The pyrolysis oil may be produced by fast pyrolysis, slow pyrolysis, or any other suitable process. The pyrolysis oil may be produced from any suitable biomass such as, for example, beech biomass. The pyrolysis oil may act as an odorant to mask the smell of the lignin in the composition. The composition may comprise about 0.1% to about 2%, or about 0.2% to about 1%, or about 0.5% pyrolysis oil by weight. The composition may comprise about 0.1% or above about 0.2% or above pyrolysis oil by weight. The composition may comprise about 2% or less or about 1% or less pyrolysis oil by weight.

In some embodiments, the composition is gasified with a gas. As used herein, “gasified” refers to introduction of a gas into the composition such that bubbles of the gas are suspended therein. The term “aerated” refers to gasifying with air or oxygen.

The gas may be selected based on the aerobic or anaerobic nature of the isolated strain(s) incorporated into the composition. In some embodiments, the gas at least partially comprises oxygen. For example, the gas may be air or relatively pure oxygen. In some embodiments, the gas may at least partially comprise carbon dioxide and/or nitrogen. Gasification may function to provide oxygen and/or other suitable gasses directly or in close proximity to the bacterial cells of the isolated strain. Gasification may promote proliferation of the bacterial cells and allow the composition to be used or stored for an extended period of time. In some embodiments, the aerated composition may have a half-life of about 20 to 30 days.

In some embodiments, the composition is gasified with nanobubbles and/or microbubbles of the gas. As used herein, “nanobubble” refers to bubbles in the nanometer range and “microbubble” refers to bubbles in the micrometer range. The nanobubbles and/or microbubbles may be introduced into the composition by any suitable means including, for example, a micro- or nanobubble nozzle or a venturi tube.

It has surprisingly been found that using a stabilized or upgraded as opposed to an otherwise unstable catholyte solution enhances the action of the compositions of the invention. Accordingly, in some embodiments, the catholyte solution is pre-treated in a system that is designed to introduce nitrogen gas into the catholyte solution, in particular in the form of nano- and/or micro-bubbles, for incorporation into a composition of the invention.

Accordingly, in some embodiments, the catholyte solution is upgraded prior to blending with the other components of the separation and recovery composition.

In some embodiments, the composition may comprise any other suitable components. For example, in some embodiments, the composition may further comprise at least one nutrient source for the live bacteria of the isolated strain.

Therefore, in some embodiments, a relatively non-toxic, inert, and sustainable composition is provided for hydrocarbon separation and/or recovery. The composition may also be relatively low cost as lignin is a waste product of pulp and paper operations that is typically discarded.

Various modifications besides those already described are possible without departing from the concepts disclosed herein. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

Although particular embodiments have been shown and described, it will be appreciated by those skilled in the art that various changes and modifications might be made without departing from the scope of the disclosure. The terms and expressions used in the preceding specification have been used herein as terms of description and not of limitation, and there is no intention in the use of such terms and expressions of excluding equivalents of the features shown and described or portions thereof.

1. A method for separating and/or recovering metals from a crushed or milled ore material comprising the metals, the method comprising:
providing a composition comprising lignin, in particular technical lignin, and at least one isolated strain of bacteria capable of producing at least one biosurfactant, and/or at least one biosurfactant produced from at least one isolated strain of bacteria capable of producing a biosurfactant;
contacting the crushed or milled ore material with the composition such that at least a portion of the metals or metal containing material separate and settle out from the rest of the crushed or milled ore material; and
recovering the separated and settled metals or metal containing material.

2. A method according to claim 1, wherein the metals are heavy metals or precious metals.

3. A method according to claim 2, wherein the heavy metals are selected from the group consisting of gold, platinum, palladium, silver, titanium, antimony, gallium, thallium and the like.

4. A method according to claim 2, wherein the precious metals are selected from the group consisting of gold, platinum, palladium, silver, osmium, iridium, rhodium, and the like.

5. A method according to claim 1, wherein the lignin comprises at least one of lignin nanoparticles and lignin microparticles.

6. A method according to claim 1, wherein the lignin includes lignin particles, at least 20% of the lignin particles being lignin nanoparticles.

7. A heavy metal separation and/or recovery composition suitable for separating and/or recovering heavy or precious metals from an ore according to the method of claim 1, in particular crushed or milled ore comprising the heavy or precious metals, the composition comprising:
lignin and at least one isolated strain of bacteria capable of producing at least one biosurfactant, and/or at least one biosurfactant produced from at least one bacteria capable of producing a biosurfactant.

8. A composition according to claim 7, wherein the lignin is technical lignin.

9. The composition of claim 8, wherein the technical lignin comprises at least one of Kraft lignin, lignosulfonates, soda lignin, organosolv lignins, steam-explosion lignin, enzymatic hydrolysis lignin, or unhydrolyzed Kraft black liquor lignin.

10. The composition of claim 7, wherein the lignin is in an aqueous suspension.

11. The composition of claim 7, wherein the lignin comprises at least one of lignin nanoparticles and lignin microparticles.

12. The composition of claim 7, wherein the lignin includes lignin particles, at least 20% of the lignin particles being lignin nanoparticles.

13. The composition of claim 7, wherein the at least one isolated bacteria strain comprises at least one isolated strain of Bacillus selected from the group consisting of Bacillus amyloliquefaciens, Bacillus licheniformis, Bacillus pumilus, Bacillus subtilis, and combinations thereof.

14. The composition of claim 7, wherein the at least one isolated strain is in the form of a liquid suspension or freeze-dried spores.

15. The composition of claim 7, further comprising a catholyte solution.

16. The composition of claim 15, wherein the catholyte solution is a stabilized or enhanced catholyte solution.

17. The composition of claim 7, further comprising at least one of a carboxylic acid or a salt or ester thereof.

18. The composition of claim 17, wherein the carboxylic acid ester comprises a methyl ester or a butyl ester, or the carboxylic acid or salt or ester thereof comprises a di-carboxylic acid or a salt or ester thereof.

19. The composition of claim 7, further comprising carbon black and/or pyrolysis oil.

20. The composition of claim 7, wherein the composition is gasified with at least one of nanobubbles and microbubbles.
